(19) United States
(12) Patent Application Publication
(10) Pub. No.: US 2026/0199330 A1
(43) Pub. Date: Jul. 16, 2026

(54) MEDICAL METHOD AND USE

(71) Applicant: ViiV Healthcare UK (No.3) Limited, Stevenage (GB)

(72) Inventors: Susan L. FORD, Durham, NC (US); Jon William Collins, Durham, NC (US)

(73) Assignee: ViiV Healthcare UK (No.3) Limited, Stevenage (GB)

(21) Appl. No.: 19/464,781

(22) Filed: Jan. 30, 2026

Related U.S. Application Data
(63) Continuation of application No. PCT/EP2025/085998, filed on Dec. 9, 2025.
(60) Provisional application No. 63/730,022, filed on Dec. 10, 2024.

(51) Int. Cl. A61K 31/4985; A61P 31/18

(52) U.S. Cl. CPC A61K 31/4985; A61P 31/18

(57) ABSTRACT

The present invention relates to Human Immunodeficiency Virus (HIV) prevention specifically, a dosing regimen for preventing HIV virus in a human.

### FIELD OF THE INVENTION

The invention relates to Human Immunodeficiency Virus (HIV) prevention. In particular, the invention relates to prevention of HIV by administering pharmaceutical compositions comprising cabotegravir.

### BACKGROUND TO THE INVENTION

Cabotegravir (CAB) is a potent integrase strand transfer inhibitor (INSTI) that possesses attributes that allow formulation and delivery as a long-acting (LA) parenteral product. Cabotegravir has been extensively studied for treatment of HIV-infection (every month [Q1M] and every 2 months [Q2M]) and prevention (Q2M) in individuals at risk of HIV acquisition, and it is currently approved for those indications in some countries.

APRETUDE® (comprising 200 mg/mL-600 mg cabotegravir, also referred to herein as CAB LA) administered intramuscularly (IM), Q2M is approved as pre-exposure prophylaxis (PrEP) to reduce the risk of sexually acquired HIV-1 infection in adolescents and adults. Long-acting therapy offers many potential advantages over daily oral regimens including infrequent dosing that decreases the daily reminder to individuals at risk of HIV infection, better tolerability, and lower likelihood to develop viral resistance due to intermittent compliance. New formulations of cabotegravir, and dosing regimens suitable to administer these, are being developed for injectable suspensions to target longer and more sustained pharmacokinetics. These formulations may achieve higher CAB concentrations than the currently approved 200 mg/mL formulation, which may allow for administration of a higher dose of cabotegravir and further extend the dosing interval between injections of cabotegravir. This may improve individuals' PrEP treatment experiences, resulting in a positive impact on prevention outcomes and acceptability.

### SUMMARY OF THE INVENTION

According to a first aspect of the invention, there is provided a method of preventing HIV infection in a human, comprising: (a) administering a first pharmaceutical composition to the human; (b) subsequently administering the first pharmaceutical composition to the human; and (c) subsequently administering a second pharmaceutical composition to the human; wherein the first pharmaceutical composition comprises about 200 mg/mL of cabotegravir, and wherein the second pharmaceutical composition comprises about 533 mg/ml of cabotegravir.

According to a second aspect of the invention, there is provided cabotegravir for use in the prevention of HIV infection in a human, the use comprising: (a) administering a first pharmaceutical composition to the human; (b) subsequently administering the first pharmaceutical composition to the human; and (c) subsequently administering a second pharmaceutical composition to the human; wherein the first pharmaceutical composition comprises about 200 mg/ml of cabotegravir, and wherein the second pharmaceutical composition comprises about 533 mg/ml of cabotegravir.

The methods of HIV prevention and use in prevention of HIV infection of the present invention may be advantageous in a number of respects. The present inventors have found new dosing strategies for PrEP. These dosing strategies enable people who would benefit from PrEP to achieve efficacious concentrations of cabotegravir plasma levels (trough plasma level (Ctau) associated with clinical efficacy: at or higher than 1.05 μg/mL for people assigned male at birth and at or higher than 1.39 μg/mL for people assigned female at birth) following the loading dose without receiving a high initiation or loading dose of cabotegravir, for example a dose of cabotegravir which may cause an undesired injection site reaction. The terms loading dose and initiation dose are used interchangeably herein to mean the same thing. The disclosed dosing schedule may allow both people who are naïve to cabotegravir and people who are already taking APRETUDE® to safely start a dosing schedule of cabotegravir which has a longer interval between doses than the currently approved regimen.

Specifically, the present invention is concerned with an initiation dosing schedule of cabotegravir which would precede regular maintenance doses of cabotegravir either for people already taking APRETUDE® or people who are naïve to cabotegravir. The present invention may allow a person who may benefit from PrEP (PWBP) to achieve cabotegravir plasma concentrations which have been shown to be efficacious without being subjected to high initiation or loading doses of cabotegravir, thus providing a safe initiation route to a maintenance dose which would then be administered at regular intervals which are given less frequently than the dosing interval of APRETUDE®. Simulations of the dosing regimen of the present invention have shown that people receiving 2 doses of APRETUDE® reach sufficient sustainable plasma exposure to CAB before they start receiving less frequent dosing of CAB than the currently approved regimen. A loading dose using APRETUDE® is beneficial as it removes the need for high loading doses of cabotegravir thus providing a safe option for the person receiving the loading dose.

A long-acting cabotegravir regimen that achieves similar trough concentrations to approved APRETUDE® but with less frequent dosing would provide similar efficacy in preventing HIV infection. In addition, a cabotegravir formulation achieving similar maximal concentrations (Cmax) as those observed with APRETUDE® is anticipated to convey the same safety. Extending the dose interval between cabotegravir injections may improve individuals' PrEP experiences, resulting in higher patient compliance, lower likelihood of resistance, and a positive impact on prevention outcomes and acceptability.

### BRIEF DESCRIPTION OF THE DRAWINGS

FIGS. 1A and 1B show the predicted Median (10th, 90th percentiles) cabotegravir (CAB) concentration time profiles by Sex at birth (male 1A, female 1B) following APRETUDE® (also described herein as CAB LA [600 mg]) initiation at day 1 and month 1 followed by CAB ULA (1600 mg-533 mg/ml, 3 mL) at month 3 and every 4 months (Q4M) from month 5. The grey line is the median CAB concentration, and dashed lines are 10th and 90th percentiles of the prediction interval.

FIGS. 2A and 2B show the predicted Median (10th, 90th percentiles) CAB concentration time profiles by Sex at birth (male 2A, female 2B) following APRETUDE® initiation at day 1 and month 1 followed by CAB ULA 1600 mg (533 mg/ml, 3 mL) at month 3 and Q4M from month 5 compared to subjects who remain on the APRETUDE® regimen. The solid grey line is the median CAB concentration for simulated subjects who start receiving CAB ULA as of Month 3. The light grey shading represents the 10th and 90th percentiles of the prediction interval for subjects who receive CAB ULA starting at Month 3. The dashed line is the median CAB concentration for simulated subjects who continue receiving APRETUDE® starting at Month 1. The dark grey shading represents 10th and 90th percentiles of the prediction interval for subjects who continue receiving APRETUDE® starting at Month 1.

FIG. 3 shows the Predicted Median (10th, 90th percentiles) CAB Concentration-Time Profile following administration of 5 doses of APRETUDE® (Day 1, then every 2 months for 4 doses starting at Month 1) followed by CAB Ultra Long-Acting (ULA) at month 9, then Q4M starting at month 11. The grey line is the median CAB concentration, and dashed lines are 10th and 90th percentiles of the prediction interval.

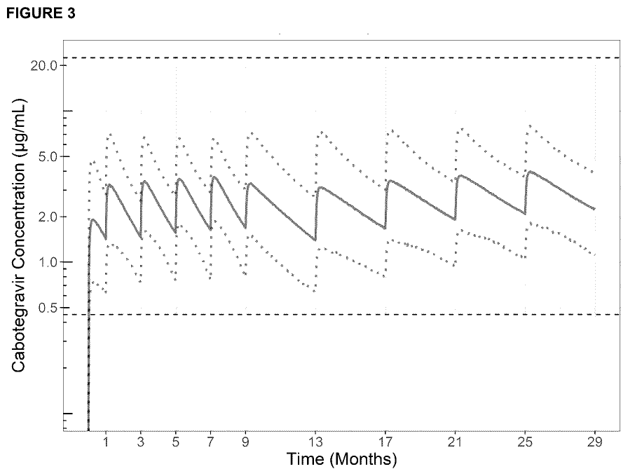

FIG. 4 shows a comparison of CAB trough concentrations following CAB LA (month 9, or M9) versus CAB ULA (month 23, or M23).

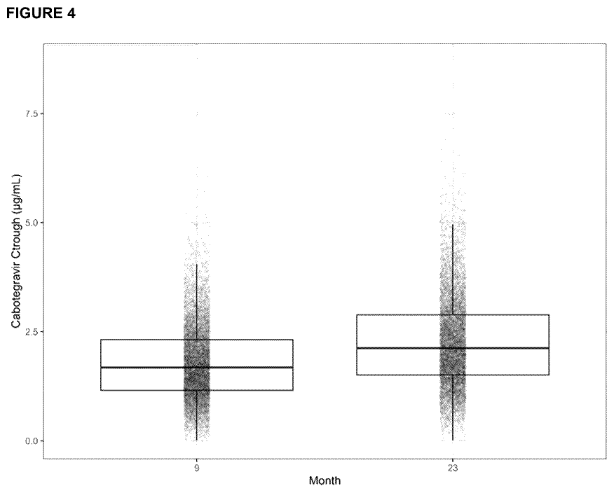

FIG. 5 shows a schematic of the study plan described in Example 4.

FIG. 6 shows a schematic of the study plan described in Example 5.

### DETAILED DESCRIPTION OF THE INVENTION

### Definitions

As used herein, the term “pharmaceutical composition” means a composition that is suitable for pharmaceutical use.

As used herein, the term “prevention” or “preventing” refers to avoidance of the stated disease in a subject who is not suffering from the stated disease.

As used herein, the term “pharmaceutically acceptable excipient” refers to an excipient that is useful in preparing a pharmaceutical composition that is generally safe and non-toxic.

As used herein, the term “treatment” or “treating” refers to alleviating the specified condition, eliminating or reducing the symptoms of the condition, slowing or eliminating the progression, invasion, or spread of the condition, and reducing or delaying the recurrence of the condition in a previously afflicted subject.

As used herein, the term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures.

As used herein, the term “lyophilization,” also known as freeze-drying or cryodesiccation, is a dehydration process which involves freezing the product without destroying the physical structure of the matter.

As used herein, the terms “lyophilized” and “freeze-dried” can be used interchangeably herein and refer to a condition and/or state of a sample, formulation, or product obtained by means of lyophilization.

As used interchangeably herein, the terms “lyophilized pharmaceutical composition” and “lyophilized composition” refer to a pharmaceutical composition in lyophilized form, as taught herein, for example a lyophilized powder.

As used herein, “reconstitution” refers to the process of restoring a dried, lyophilized, dehydrated, or concentrated matter to its original or liquid state by adding a solvent to the lyophilized matter, allowing the lyophilized matter to rehydrate, followed by agitating the mixture of the solvent and lyophilized matter. The reconstituted matter may be part of a product, formulation, sample, raw material, or any biological material but is certainly not limited to matter falling under the common definition of these terms. Reconstitution can be assessed visually with the naked eye. The lyophilized matter is deemed reconstituted when a homogeneous suspension is observed. In particular, a suspension with a cloudy appearance is considered suitably reconstituted.

As used herein, the term “subject” or “patient” refers to a human.

As used herein, the term “assigned female at birth” or “AFAB” refers to a human whose sex assigned at birth was female. The human may identify as (non-exhaustively) male, female or non-binary.

As used herein, the term “assigned male at birth” or “AMAB” refers to a human whose sex assigned at birth was male. The human may identify as (non-exhaustively) male, female or non-binary.

As used herein, the term “diameter” refers to a spherical volume equivalent diameter. As used herein, the term “Cmax” refers to maximum observed plasma concentration. As used herein, the term “Ctau” refers to trough plasma concentration, i.e. the concentration reached immediately before the next dose is administered.

As used herein the term “tmax” refers to time of maximum observed plasma concentration (tmax) of a substance after administration of that substance.

As used herein the term “Area under the curve” or “AUC” refers to area under the concentration (of substance in plasma)—time curve. AUC can be a measure of the integral of the instantaneous concentrations during a time interval and has the units mass*time/volume. AUC is typically calculated by the trapezoidal method (e.g., linear, linear-log). AUC is usually given for the time interval zero to infinity (AUC (0-inf)), and other time intervals are indicated (for example AUC (t1,t2) where t1 and t2 are the starting and finishing times for the interval).

As used herein, the term “about” generally means±5%, ±10%, ±15%, or ±20% of the numerical value of the number with which it is being used. In an embodiment, the term “about” means±10% of the numerical value of the number with which it is being used.

As used herein, the term “cabotegravir naïve” refers to a human who has not previously been taking APRETUDE® for HIV PrEP.

### DESCRIPTION OF THE EMBODIMENTS

In a first aspect, the present invention provides a method of preventing HIV infection in a human, comprising: (a) administering a first pharmaceutical composition to the human; (b) subsequently administering the first pharmaceutical composition to the human; and (c) subsequently administering a second pharmaceutical composition to the human; wherein the first pharmaceutical composition comprises about 200 mg/ml of cabotegravir, and wherein the second pharmaceutical composition comprises about 533 mg/ml of cabotegravir.

The present invention provides a method of preventing HIV infection in a human, comprising: (a) administering a first pharmaceutical composition to the human; (b) subsequently administering the first pharmaceutical composition to the human; and (c) subsequently administering a second pharmaceutical composition to the human; wherein the first pharmaceutical composition comprises 200 mg/ml of cabotegravir, and wherein the second pharmaceutical composition comprises 533 mg/mL of cabotegravir.

Methods of the invention prevent HIV infection in humans. In an embodiment the human is a person who would benefit from pre-exposure prophylaxis (PrEP) (PWBP). PrEP is the use of antiretroviral medication by HIV-negative people to reduce the risk of HIV acquisition. Prevention of HIV infection and PrEP are used interchangeably herein. A person who would benefit from PrEP (PWBP) is a person who is HIV-negative and at risk of HIV acquisition. A PWBP may be exposed to HIV infection through sex or drug use.

The method of the invention involves a step (a) of administering a first pharmaceutical composition to the human. The first pharmaceutical composition comprises about 200 mg/mL of cabotegravir. In an embodiment, the first pharmaceutical composition comprises 200 mg/ml of cabotegravir.

### Cabotegravir

Cabotegravir (3S, 11aR)-N-((2,4-difluorophenyl)methyl)-6-hydroxy-3-methyl-5,7-dioxo-2,3,5, 11,11a-hexahydro(1,3)oxazolo(3,2-a)pyrido(1,2-d)pyrazine-8-carboxamide is described in U.S. Pat. No. 8,129,385 in example Z-9 which example is incorporated herein by reference. Cabotegravir is an integrase strand transfer inhibitor (INSTI) that exhibits subnanomolar potency and antiviral activity against a broad range of HIV-1 strains. Oral administration of cabotegravir has exhibited acceptable safety and tolerability profiles, a long half-life, and few drug-drug interactions. Cabotegravir has been demonstrated to be efficacious in treatment and prevention of HIV both in oral and parenteral dosage forms, see for instance, Margolis D A, Brinson C C, Eron J J, et al. 744 and Rilpivirine as Two Drug Oral Maintenance Therapy: LAI116482 (LATTE) Week 48 Results. 21^{st} Conference on Retroviruses and Opportunistic Infections (CROI); Mar. 3-6, 2014; Boston, MA, Margolis D A, Podzamczer D, Stellbrink H-J, et al. Cabotegravir+Rilpivirine as Long-Acting Maintenance Therapy: LATTE-2 Week 48 Results, 21^{st} International AIDS Conference; Jul. 18-22, 2016; Durban, South Africa, Abstract THAB0206LB. Levin: Conference reports for National AIDS Treatment Advocacy Project (NATAP); 2016, and Markowitz M, Frank I, Grant R, et al. ÉCLAIR: Phase 2A Safety and PK Study of Cabotegravir LA in HIV-Uninfected Men. Abstract presented at 23^{rd} CROI; Feb. 22-25, 2016; Boston, MA. Cabotegravir has been approved by the FDA, as well as many other health authorities, for long-acting prevention of HIV infection dosed every two months; and in combination with rilpivirine for long-acting treatment of HIV infection dosed once a month or once every two months.

Cabotegravir is represented by Compound A:

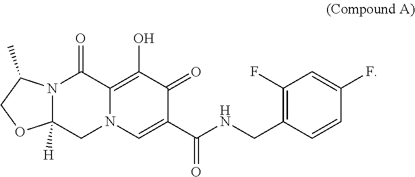 [SMILES: C[C@H]1CO[C@@H]2Cn3cc(C(=O)NCc4ccc(F)cc4F)c(=O)c(O)c3C(=O)N12]

### First Pharmaceutical Composition

The first pharmaceutical composition comprises about 200 mg/ml of cabotegravir. In an embodiment, the first pharmaceutical composition comprises about 200 mg/ml of cabotegravir and at least one pharmaceutically acceptable excipient. In an embodiment, the first pharmaceutical composition comprises 200 mg/ml of cabotegravir. In an embodiment, the first pharmaceutical composition comprises 200 mg/mL of cabotegravir and at least one pharmaceutically acceptable excipient. In an embodiment the first pharmaceutical composition is an injectable suspension. In an embodiment the first pharmaceutical composition comprises about 600 mg of cabotegravir. In an embodiment, the first pharmaceutical composition comprises about 600 mg of cabotegravir and at least one pharmaceutically acceptable excipient. In another embodiment of the invention, the first pharmaceutical composition comprises 600 mg of cabotegravir. In another embodiment, the first pharmaceutical composition comprises 600 mg of cabotegravir and at least one pharmaceutically acceptable excipient.

In an embodiment the first pharmaceutical composition further comprises polyethylene glycol (PEG), which is a pharmaceutically acceptable excipient. In an embodiment the PEG is PEG 3350. In an embodiment, the first pharmaceutical composition further comprises polysorbate 20, which is a pharmaceutically acceptable excipient. In a further embodiment, the first pharmaceutical composition further comprises mannitol, which is a pharmaceutically acceptable excipient. In an embodiment, the first pharmaceutical composition comprises 600 mg of cabotegravir, PEG 3350, polysorbate 20, and mannitol. In an embodiment, the first pharmaceutical composition is as described in WO2012/037320 which is incorporated herein by reference.

In an embodiment the first pharmaceutical composition is suitable for use as injectable. In an embodiment, the first pharmaceutical composition is an injectable suspension. In an embodiment, the first pharmaceutical composition is an injectable suspension suitable for gluteal administration.

In an embodiment the first pharmaceutical composition is APRETUDE®. APRETUDE®, also referred to herein as Apretude, is an approved extended release cabotegravir injectable suspension used to prevent HIV infection. Apretude comprises 3 mL of the composition shown in Table 1.

TABLE 1

| Ingredient | Concentration | |
|---|---|---|
| Cabotegravir | 200 | mg/mL |
| Mannitol | 35 | mg/mL |
| Polyethylene glycol (PEG) 3350 | 20 | mg/mL |
| Polysorbate 20 | 20 | mg/mL |

In an embodiment, the first pharmaceutical composition is administered by any suitable means. In an embodiment the first pharmaceutical composition is administered intramuscularly. In this embodiment, the first pharmaceutical composition may be administered in a gluteus muscle of the human. In a specific embodiment, the first pharmaceutical composition is administered at the ventrogluteal or dorsogluteal site.

### Second Pharmaceutical Composition

The second pharmaceutical composition comprises about 533 mg/mL of cabotegravir. In an embodiment, the second pharmaceutical composition comprises about 533 mg/ml of cabotegravir and at least one pharmaceutically acceptable excipient. In an embodiment, the second pharmaceutical composition comprises 533 mg/ml of cabotegravir. In an embodiment, the second pharmaceutical composition comprises 533 mg/ml of cabotegravir and at least one pharmaceutically acceptable excipient. In an embodiment the second pharmaceutical composition comprises about 1600 mg of cabotegravir. In an embodiment the second pharmaceutical composition comprises about 1600 mg of cabotegravir and at least one pharmaceutically acceptable excipient. In an embodiment the second pharmaceutical composition comprises 1600 mg of cabotegravir. In an embodiment the second pharmaceutical composition comprises 1600 mg of cabotegravir and at least one pharmaceutically acceptable excipient.

In an embodiment, the second pharmaceutical composition used in the invention comprises polysorbate 80 (PS80), which is a pharmaceutically acceptable excipient. PS80 (IUPAC name: polyoxyethylene (20) sorbitan monooleate; CAS No. 9005-65-6) is a nonionic surfactant and emulsifier derived from polyethoxylated sorbitan and oleic acid. The hydrophilic groups in PS80 are polyethers also known as polyoxyethylene groups, which are polymers of ethylene oxide. In the nomenclature of polysorbates, the numeric designation following “polysorbate” (e.g., “polysorbate 80”) refers to the lipophilic group, in this case, the oleic acid. The structure of PS80 is provided by formula (I):

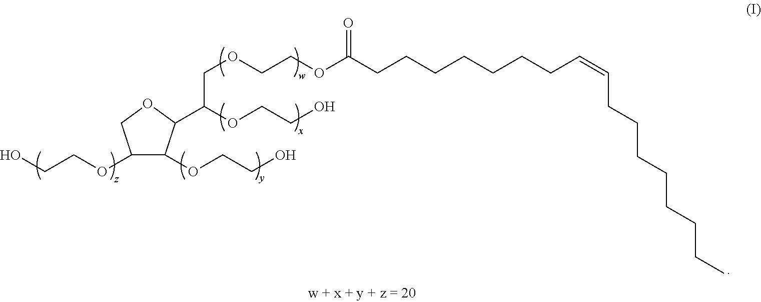

In an embodiment, the second pharmaceutical composition contains from about 0.1 mg/mL to about 150 mg/ml of PS80. In a further embodiment, the second pharmaceutical composition contains from about 1 mg/ml to about 80 mg/mL, from about 2 mg/mL to about 40 mg/mL, from about 2.5 mg/ml to about 6 mg/mL, or from about 2.5 mg/mL to about 5 mg/ml of PS80. In an embodiment, the second pharmaceutical composition contains about 5 to about 6 mg/mL of PS80. In an embodiment, the second pharmaceutical composition contains about 5.33 mg/ml of PS80.

In an embodiment, a weight ratio of the PS80 to cabotegravir in the second pharmaceutical composition is in a range of from 1:10 to 1:400. In another embodiment, the weight ratio of the PS80 to cabotegravir is in a range of from 1:50 to 1:200. In another embodiment, the weight ratio of the PS80 to cabotegravir is in a range of from 1:100 to 1:150. In another embodiment, the weight ratio of the PS80 to cabotegravir is about 1:100.

In an embodiment, the second pharmaceutical composition further comprises sodium carboxymethylcellulose (CMC), which is a pharmaceutically acceptable excipient. In an embodiment, the second pharmaceutical composition contains about 0.1 to about 150 mg/mL of sodium CMC In a further embodiment, the second pharmaceutical composition contains about 1 mg/mL to about 25 mg/mL, from about 2 mg/ml to about 15 mg/mL, from about 2 mg/mL to about 10 mg/mL, or from about 3 mg/mL to about 10 mg/ml of sodium CMC. In an embodiment, the second pharmaceutical composition comprises about 6 to about 7 mg/ml of sodium CMC.

In an embodiment, a weight ratio of sodium CMC to cabotegravir is in a range of from 1:10 to 1:400. In another embodiment, the weight ratio of sodium CMC to cabotegravir is in a range of from 1:40 to 1:200. In another embodiment, the weight ratio of sodium CMC to cabotegravir is in a range of from 1:70 to 1:120. In another embodiment, the weight ratio of sodium CMC to cabotegravir is about 1:80. In another embodiment, the weight ratio of sodium CMC to cabotegravir is 1:80.

In an embodiment, the second pharmaceutical composition of the invention comprises mannitol. In an embodiment, the second pharmaceutical composition contains about 0.1 to about 250 mg/mL of mannitol. In a further embodiment, the second pharmaceutical composition contains about 1 mg/mL to about 150 mg/mL, from about 10 mg/ml to about 125 mg/mL, from about 15 mg/mL to about 60 mg/mL, from about 15 mg/mL to about 50 mg/mL, from about 20 mg/mL to about 50 mg/mL, or from about 20 mg/ml to about 40 mg/ml of mannitol.

In an embodiment, a weight ratio of the mannitol to cabotegravir is in a range of from 1:1 to 1:100. In another embodiment, the weight ratio of the mannitol to cabotegravir is in a range of from 1:5 to 1:50. In another embodiment, the weight ratio of the mannitol to cabotegravir is in a range of from 1:8 to 1:25. In another embodiment, the weight ratio of the mannitol to cabotegravir is about 1:11.4. In another embodiment, the weight ratio of the mannitol to cabotegravir is 1:11.4.

In an embodiment the second pharmaceutical composition comprises polysorbate 80 and sodium carboxymethylcellulose (CMC). In an embodiment the second pharmaceutical composition comprises polysorbate 80, sodium carboxymethylcellulose (CMC) and mannitol. In an embodiment the second pharmaceutical composition comprises polysorbate 80, sodium carboxymethylcellulose (CMC) and mannitol and the ratio of cabotegravir:polysorbate 80:sodium CMC is 400:4:5.

The second pharmaceutical composition of the present invention comprises particles of crystalline cabotegravir. In an embodiment, cabotegravir particles of the second pharmaceutical composition have an X50 value greater than or equal to 2.5 μm and less than or equal to 10 μm (i.e., 2.5 μm≤×50≤10 μm). Particle size distribution may be measured by any suitable method, for example, by laser diffraction. Laser diffraction is sensitive to the volume of a particle and provides a volume-average particle size, which is equivalent to the weight-average particle size if the density is constant. It will be apparent to those skilled in the art that the results of the particle size distribution determination by one technique can be correlated with that from another technique, for example on an empirical basis by routine experimentation. Alternatively, particle size distribution can be determined by microscopy, in particular electron microscopy or scanning electron microscopy.

As used herein, X50 (or “the X50 value”) is the cabotegravir particle diameter, in microns, at which 50% by volume of the cabotegravir particles have a smaller diameter and 50% by volume have a larger diameter, also known as the mass median diameter (MMD) or the median of the particle size distribution by volume.

In an embodiment of the invention, the X50 of the second pharmaceutical composition is between (and including) 3 and 8.5 μm. In an embodiment of the invention, the X50 of the second pharmaceutical composition is between (and including) 4 and 6 μm.

In an embodiment, the second pharmaceutical composition is reconstituted from a lyophilized composition. Lyophilization comprises at least a freezing step and a sublimation step. Lyophilization may be used in the manufacturing of pharmaceutical products and intermediates thereof. During freezing, the material is cooled to a temperature wherein the solid, liquid, and gas phases of the material may exist. Active pharmaceutical product ingredients (APIs) may be lyophilized to achieve chemical and physical stability allowing room temperature storage. This is different from a conventional method that evaporates water using heat. Advantages of lyophilization may include, but are not limited to, enhanced stability of a dry powder, the removal of water without excessive heating of the product, and enhanced product stability in a dry state.

In an exemplary method of preparing a lyophilized formulation of the invention, micronized cabotegravir free acid is packaged in antistatic linear low-density polyethylene bags. The packaged cabotegravir is enclosed in a corrugated plastic box and gamma irradiated as a bioburden reduction step and referred to as gamma irradiated cabotegravir. Gamma irradiated cabotegravir is dispersed in a filtered aqueous vehicle comprising a stabilizer (e.g., sodium CMC), a tonicity agent (e.g., mannitol), and a wetting agent (e.g., PS80). The resulting suspension is filled into washed, sterilized/depyrogenated 10 mL Type I clear glass vials. Container materials are then processed: vials are depyrogenated by dry heat, and stoppers and overseals are sterilized by steam. Product vials are lyophilized, backflushed with nitrogen, sealed with halobutyl rubber stoppers and secured by an aluminum overseal. The sealed vials are terminally sterilized by gamma irradiation.

In an embodiment, the lyophilized pharmaceutical composition is a suspension. The lyophilized pharmaceutical composition advantageously suspends when reconstituted in an aqueous or non-aqueous solution, that is, all or substantially all, such as at least 90 percent, at least 95 percent, at least 96 percent, at least 97 percent, at least 98 percent, at least 99 percent, at least 99.5 percent or 100 percent of the lyophilized pharmaceutical composition is suspended when reconstituted.

Reconstitution can be assessed visually with the naked eye. The lyophilized matter is deemed reconstituted when a homogeneous suspension is observed. In particular, a suspension with a cloudy appearance is considered suitably reconstituted.

It will be apparent to those skilled in the art that pharmaceutical compositions described herein may be reconstituted in an aqueous or non-aqueous solution to a desired concentration.

In an embodiment, the second pharmaceutical composition, after lyophilization and reconstitution, is as described in Table 2:

TABLE 2

| Ingredient | Concentration (mg/mL) |
|---|---|
| Cabotegravir | 450-667 (X10 = 1.4-3.3 μm; X50 = |
| | 3.4-7.5 μm; X90 = 7.6-21.3 μm) |
| Polysorbate 80 | 2.5-5 |
| Sodium CMC | 3.1-6.9 |
| Mannitol | 22-50 |

In another embodiment, the second pharmaceutical composition, after lyophilization and reconstitution with 1.1 mL water, is as described in Table 3. In an embodiment, the second pharmaceutical composition comprises 3 mL of the composition in Table 3.

TABLE 3

| Ingredient | Concentration (mg/mL) |
|---|---|
| Cabotegravir | 533 (X50 = 4-6 μm) |
| Polysorbate 80 | 5.3 |
| Sodium CMC | 6.7 |
| Mannitol | 46.6 |

In an embodiment, the second pharmaceutical composition is as described in Table 4.

TABLE 4

| Ingredient | Amount (Mg) |
|---|---|
| Cabotegravir | 1600 (X50 = 4-6 μm) |
| Polysorbate 80 | 20 |
| Sodium CMC | 16 |
| Mannitol | 140 |

### Method of Prevention

The present invention provides a method of preventing HIV infection in a human, comprising: (a) administering a first pharmaceutical composition to the human; (b) subsequently administering the first pharmaceutical composition to the human; and (c) subsequently administering the second pharmaceutical composition to the human; wherein the first pharmaceutical composition comprises about 200 mg/mL of cabotegravir, and wherein the second pharmaceutical composition comprises about 533 mg/mL of cabotegravir.

In an embodiment of the invention, both the first and second pharmaceutical compositions are suitable for injection, and the step of administering is via intramuscular injection. In an embodiment, the administering is via intramuscular administration into a gluteus muscle, for example, the administering is via intramuscular administrating into the gluteus maximum, gluteus medius or gluteus minimus. In an embodiment, the intramuscular administration is administered by a ventrogluteal approach or a dorsogluteal approach. In embodiments of the invention, the intramuscular injections are rotated between left and right gluteal muscles at alternative injections in order to extend the interval between injections at the same site. For example, in this embodiment, the administering in step (b) may be injected into a right gluteal muscle and the administering in step (c) may be injected into a left gluteal muscle. If further injections occur these would then be rotated such that the human is not receiving 2 injections into the same site at concurrent administration events.

In an embodiment, the method comprises (b) administering the first pharmaceutical composition to the human; and (c) subsequently administering the second pharmaceutical composition to the human; wherein step (c) occurs 2 months ±7 days after step (b). In this embodiment, the ±7 days after the 2-month period may be referred to as the grace period, i.e. the scheduled injection may be taken up to 7 days either side of the 2-month date. In an embodiment, step (c) occurs 2 months after step (b). In an embodiment, the first composition comprises 3 mL of the composition shown in Table 1, the second composition comprises 3 mL of the composition shown in Table 3, and step (c) occurs 2 months ±7 days after step (b).

In an embodiment, the method of the invention further comprises a step (d) of administering to the human the second pharmaceutical composition 2 months ±7 days after step (c). In this embodiment, the method comprises:
(a) administering a first pharmaceutical composition to the human;
(b) administering the first pharmaceutical composition to the human;
(c) subsequently administering the second pharmaceutical composition to the human; and
(d) administering to the human the second pharmaceutical composition 2 months ±7 days after step (c).

In this embodiment, the ±7 days after the 2-month period may be referred to as the grace period i.e. the scheduled injection may be taken up to 7 days either side of the 2-month date. In an embodiment, in step (d) the administering occurs 2 months after step (c).

In an embodiment, the method further comprises a step (e) administering to the human the second pharmaceutical composition 4 months ±14 days after step (d). In this embodiment, the method comprises:
(a) administering a first pharmaceutical composition to the human;
(b) administering the first pharmaceutical composition to the human;
(c) subsequently administering the second pharmaceutical composition to the human;
(d) administering to the human the second pharmaceutical composition 2 months ±7 days after step (c); and
(e) administering to the human the second pharmaceutical composition 4 months ±14 days after step (d).

In this embodiment, the ±14 days after the 4-month period may be referred to as the grace period, i.e. the scheduled injection may be taken up to 14 days either side of the 4-month date. In an embodiment, in step (e) the administering occurs 4 months after step (d).

In an embodiment, the method further comprises a step (f) of administering the second pharmaceutical composition every 4 months ±14 days thereafter. In this embodiment the method comprises:
(a) administering a first pharmaceutical composition to the human;
(b) administering the first pharmaceutical composition to the human;
(c) subsequently administering the second pharmaceutical composition to the human;
(d) administering to the human the second pharmaceutical composition 2 months ±7 days after step (c);
(e) administering to the human the second pharmaceutical composition 4 months ±14 days after step (d); and
(f) administering the second pharmaceutical composition every 4 months ±14 days thereafter.

In this embodiment, the ±14 days after the 4-month period may be referred to as the grace period, i.e. the scheduled injection may be taken up to 14 days either side of the 4-month date. In an embodiment, the administering in step (f) occurs every 4 months thereafter.

In an embodiment of the invention prior to step (a), the human has never previously been administered the first pharmaceutical composition. In these embodiments, the human may be referred to as ‘cabotegravir naïve’. The human may have previously been on a different PrEP regimen, for example an oral PrEP regimen. In an embodiment, prior to step (a), the human has never previously been administered Apretude. In these embodiments (i.e., cabotegravir-naïve human), step (b) occurs 1 month ±7 days after step (a). In an embodiment, prior to step (a), the human has never previously been administered Apretude, and step (b) occurs 1 month ±7 days after step (a). In an embodiment, prior to step (a), the human has never previously been administered Apretude, and step (b) occurs 1 month after step (a).

In an embodiment of the invention the method comprises the steps of:
(a) administering the first pharmaceutical composition to the human;
(b) 1 month ±7 days later administering the first pharmaceutical composition to the human; and
(c) 2 months ±7 days later administering the second pharmaceutical composition to the human; wherein prior to step (a) the human has never previously been administered the first pharmaceutical composition; and wherein the first pharmaceutical composition is Apretude.

In an alternative embodiment of the invention, step (b) occurs 2 months ±7 days after step (a). In this embodiment the method comprises a step (a) of administering the first pharmaceutical composition to the human; (b) 2 months ±7 days later administering the first pharmaceutical composition to the human; and (c) subsequently administering the second pharmaceutical composition to the human. In some embodiments, the first pharmaceutical composition is Apretude and the method comprises administering Apretude to the human and 2 months ±7 days later administering Apretude to the human again; and (c) subsequently administering the second pharmaceutical composition to the human. In embodiments of the invention, step (b) occurs 2 months after step (a). In embodiments of the invention step (b) occurs 2 months ±7 days after step (a) or 2 months after step (a), and prior to step (a) the human has been administered the first pharmaceutical composition at least 6 times, at least 5 times, at least 4 times at least 3 times, at least 2 times or at least once. In these embodiments the human has been administered the first pharmaceutical composition at least 6 times, at least 5 times, at least 4 times, at least 3 times at least 2 times or at least once, each time 2 months ±7 days apart, for example 2 months apart. In embodiments of the invention step (b) occurs 2 months ±7 days after step (a) or 2 months after step (a), and prior to step (a) the human has been administered the first pharmaceutical composition at least once. In embodiments of the invention, step (b) occurs 2 months after step (a). In embodiments of the invention, step (b) occurs 2 months ±7 days after step (a) or 2 months after step (a), and prior to step (a) the human has been administered the first pharmaceutical composition at least 3 times. In this embodiment the human has been administered the first pharmaceutical composition at least 3 times, each time 2 months ±7 days apart, for example 2 months apart. In an embodiment, prior to step (a), the human has been administered the first pharmaceutical composition at least 3 times, each time 2 months ±7 days apart for example 2 months apart. In these embodiments, the human may already be taking Apretude every 2 months and switches from Apretude to taking the second pharmaceutical composition of the present invention (step (b)). In these embodiments the human is taking Apretude every 2 months and then at their regular 2-monthly injection date switches to the second pharmaceutical composition.

In an embodiment of the invention, the method comprises the steps of:
(a) administering the first pharmaceutical composition to the human;
(b) 2 months ±7 days later administering the first pharmaceutical composition to the human; and
(c) subsequently administering the second pharmaceutical composition to the human; wherein, prior to step (a), the human has been administered the first pharmaceutical composition at least once and wherein the first pharmaceutical composition is Apretude.

In an embodiment of the invention, the method comprises the steps of:
(a) administering the first pharmaceutical composition to the human;
(b) 2 months ±7 days later administering the first pharmaceutical composition to the human; and
(c) subsequently administering the second pharmaceutical composition to the human; wherein, prior to step (a), the human has been administered the first pharmaceutical composition at least 3 times, each time 2 months ±7 days apart; and wherein the first pharmaceutical composition is Apretude.

In embodiments of the invention the human is tested for HIV infection prior to step (a) and/or step (b). Testing for HIV infection may be done by any suitable means.

### Exemplary Embodiments

In an embodiment the method comprises the steps of:
(a) administering the first pharmaceutical composition to the human;
(b) 1 month ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter.

In this embodiment, the first pharmaceutical composition is Apretude and, prior to step (a) the human has never previously been administered Apretude.

In an embodiment the method comprises the steps of:
(a) administering the first pharmaceutical composition to the human;
(b) 1 month ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter.

In this embodiment, the first pharmaceutical composition is Apretude and, prior to step (a) the human has never previously been administered Apretude, and the second pharmaceutical composition comprises 3 mL of the composition in Table 3.

In an alternative embodiment, the method comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
wherein, prior to step (a), the human has been administered the first pharmaceutical composition at least once and wherein the first pharmaceutical composition is Apretude.

In an alternative embodiment, the method comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
wherein, prior to step (a), the human has been administered the first pharmaceutical composition at least once and wherein the first pharmaceutical composition is Apretude and the second pharmaceutical composition comprises 3 mL of the composition in Table 3.

In an alternative embodiment, the method comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
wherein, prior to step (a), the human has been administered the first pharmaceutical composition at least 3 times, each time 2 months ±7 days apart; and wherein the first pharmaceutical composition is Apretude and the second pharmaceutical composition comprises 3 mL of the composition in Table 3.

### Cabotegravir for Use in the Prevention of HIV Infection

In a second aspect, the present invention provides cabotegravir for use in the prevention of HIV infection in a human, the use comprising: (a) administering a first pharmaceutical composition to the human; (b) subsequently administering the first pharmaceutical composition to the human; and (c) subsequently administering a second pharmaceutical composition to the human; wherein the first pharmaceutical composition comprises about 200 mg/ml of cabotegravir, and wherein the second pharmaceutical composition comprises about 533 mg/ml of cabotegravir.

Cabotegravir, the human, the first pharmaceutical composition and the second pharmaceutical composition are as defined in the first aspect.

The present invention provides cabotegravir for use in the prevention of HIV infection in a human, comprising: (a) administering a first pharmaceutical composition to the human; (b) subsequently administering the first pharmaceutical composition to the human; and (c) subsequently administering the second pharmaceutical composition to the human; wherein the first pharmaceutical composition comprises about 200 mg/ml of cabotegravir, and wherein the second pharmaceutical composition comprises about 533 mg/ml of cabotegravir.

In an embodiment of the invention, both the first and second pharmaceutical compositions are suitable for injection, and the step of administering is via intramuscular injection. In an embodiment, the administering is via intramuscular administration into a gluteus muscle, for example, the administering is via intramuscular administrating into the gluteus maximum, gluteus medius or gluteus minimus. In an embodiment, the intramuscular administration is administered by a ventrogluteal approach or a dorsogluteal approach. In embodiments of the invention, the intramuscular injections are rotated between the a left and right gluteal muscle at alternative injections in order to extend the interval between injections at the same site. For example, in this embodiment, the administering in step (b) may be injected into the a right gluteal muscle and the administering in step (c) may be injected into the a left gluteal muscle. If further injections occur these would then be rotated such that the human is not receiving 2 injections into the same site at concurrent administration events.

In an embodiment, the cabotegravir for use comprises (b) administering the first pharmaceutical composition to the human; and (c) subsequently administering the second pharmaceutical composition to the human; wherein step (c) occurs 2 months ±7 days after step (b). In this embodiment, the ±7 days after the 2-month period may be referred to as the grace period, i.e. the scheduled injection may be taken up to 7 days either side of the 2-month date. In an embodiment, step (c) occurs 2 months after step (b). In an embodiment, the first composition comprises 3 mL of the composition shown in Table 1, the second composition comprises 3 mL of the composition shown in Table 3, and step (c) occurs 2 months ±7 days after step (b).

In an embodiment, the cabotegravir for use of the invention further comprises a step (d) of administering to the human the second pharmaceutical composition 2 months ±7 days after step (c). In this embodiment, the method comprises:
(a) administering a first pharmaceutical composition to the human;
(b) administering the first pharmaceutical composition to the human;
(c) subsequently administering the second pharmaceutical composition to the human; and
(d) administering to the human the second pharmaceutical composition 2 months ±7 days after step (c).

In this embodiment, the ±7 days after the 2-month period may be referred to as the grace period i.e. the scheduled injection may be taken up to 7 days either side of the 2-month date. In an embodiment, in step (d) the administering occurs 2 months after step (c).

In an embodiment, the cabotegravir for use further comprises a step (e) administering to the human the second pharmaceutical composition 4 months ±14 days after step (d). In this embodiment, the method comprises:
(a) administering a first pharmaceutical composition to the human;
(b) administering the first pharmaceutical composition to the human;
(c) subsequently administering the second pharmaceutical composition to the human;
(d) administering to the human the second pharmaceutical composition 2 months ±7 days after step (c); and
(e) administering to the human the second pharmaceutical composition 4 months ±14 days after step (d).

In this embodiment, the ±14 days after the 4-month period may be referred to as the grace period, i.e. the scheduled injection may be taken up to 14 days either side of the 4-month date. In an embodiment, in step (e) the administering occurs 4 months after step (d).

In an embodiment, the cabotegravir for use further comprises a step (f) of administering the second pharmaceutical composition every 4 months ±14 days thereafter. In this embodiment the method comprises:
(a) administering a first pharmaceutical composition to the human; (b) administering the first pharmaceutical composition to the human;
(c) subsequently administering the second pharmaceutical composition to the human;
(d) administering to the human the second pharmaceutical composition 2 months ±7 days after step (c);
(e) administering to the human the second pharmaceutical composition 4 months ±14 days after step (d); and
(f) administering the second pharmaceutical composition every 4 months ±14 days thereafter.

In this embodiment, the ±14 days after the 4-month period may be referred to as the grace period, i.e. the scheduled injection may be taken up to 14 days either side of the 4-month date. In an embodiment, the administering in step (f) occurs every 4 months thereafter.

In an embodiment of the invention prior to step (a), the human has never previously been administered the first pharmaceutical composition. In these embodiments, the human may be referred to as ‘cabotegravir naïve’. The human may have previously been on a different PrEP regimen, for example an oral PrEP regimen. In an embodiment, prior to step (a), the human has never previously been administered Apretude. In these embodiments (i.e., cabotegravir-naïve human), step (b) may occur 1 month ±7 days after step (a). In an embodiment, prior to step (a), the human has never previously been administered Apretude, and step (b) occurs 1 month ±7 days after step (a). In an embodiment, prior to step (a), the human has never previously been administered Apretude, and step (b) occurs 1 month after step (a).

In an embodiment of the invention the cabotegravir for use comprises:
(a) administering the first pharmaceutical composition to the human;
(b) 1 month ±7 days later administering the first pharmaceutical composition to the human; and
(c) 2 months ±7 days later administering the second pharmaceutical composition to the human; wherein prior to step (a) the human has never previously been administered the first pharmaceutical composition; and wherein the first pharmaceutical composition is Apretude.

In an embodiment of the invention, step (b) occurs 2 months ±7 days after step (a). In this embodiment the method comprises a step (a) of administering the first pharmaceutical composition to the human; (b) 2 months ±7 days later administering the first pharmaceutical composition to the human; and (c) subsequently administering the second pharmaceutical composition to the human. In some embodiments, the first pharmaceutical composition is Apretude and the method comprises administering Apretude to the human and 2 months ±7 days later administering Apretude to the human again; and (c) subsequently administering the second pharmaceutical composition to the human. In embodiments of the invention, step (b) occurs 2 months after step (a). In embodiments of the invention step (b) occurs 2 months ±7 days after step (a) or 2 months after step (a), and prior to step (a) the human has been administered the first pharmaceutical composition at least 6 times, at least 5 times, at least 4 times at least 3 times, at least 2 times or at least once. In these embodiments the human has been administered the first pharmaceutical composition at least 6 times, at least 5 times, at least 4 times, at least 3 times at least 2 times or at least once, each time 2 months ±7 days apart, for example 2 months apart. In embodiments of the invention step (b) occurs 2 months ±7 days after step (a) or 2 months after step (a), and prior to step (a) the human has been administered the first pharmaceutical composition at least once. In embodiments of the invention, step (b) occurs 2 months after step (a). In embodiments of the invention step (b) occurs 2 months ±7 days after step (a) or 2 months after step (a), and prior to step (a) the human has been administered the first pharmaceutical composition at least 3 times. In this embodiment the human has been administered the first pharmaceutical composition at least 3 times, each time 2 months ±7 days apart, for example 2 months apart. In an embodiment, prior to step (a), the human has been administered the first pharmaceutical composition at least 3 times, each time 2 months ±7 days apart for example 2 months apart. In these embodiments, the human may already be taking Apretude every 2 months and switches from Apretude to taking the second pharmaceutical composition of the present invention (step (b)). In these embodiments the human is taking Apretude every 2 months and then at their regular 2-monthly injection date switches to the second pharmaceutical composition.

In an embodiment of the invention, the cabotegravir for use comprises the steps of:
(a) administering the first pharmaceutical composition to the human;
(b) 2 months ±7 days later administering the first pharmaceutical composition to the human; and
(c) subsequently administering the second pharmaceutical composition to the human; wherein, prior to step (a), the human has been administered the first pharmaceutical composition at least once and wherein the first pharmaceutical composition is Apretude.

In an embodiment of the invention, the method comprises the steps of:
(a) administering the first pharmaceutical composition to the human;
(b) 2 months ±7 days later administering the first pharmaceutical composition to the human; and
(c) subsequently administering the second pharmaceutical composition to the human; wherein, prior to step (a), the human has been administered the first pharmaceutical composition at least 3 times, each time 2 months ±7 days apart; and wherein the first pharmaceutical composition is Apretude.

In embodiments of the invention the human is tested for HIV infection prior to step (a) and/or step (b). Testing for HIV infection may be done by any suitable means.

### Exemplary Embodiments

In an embodiment the cabotegravir for use comprises the steps of:
(a) administering the first pharmaceutical composition to the human;
(b) 1 month ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter.

In this embodiment, the first pharmaceutical composition is Apretude and, prior to step (a) the human has never previously been administered Apretude,

In an embodiment the cabotegravir for use comprises the steps of:
(a) administering the first pharmaceutical composition to the human;
(b) 1 month ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter.

In this embodiment, the first pharmaceutical composition is Apretude and, prior to step (a) the human has never previously been administered Apretude, and the second pharmaceutical composition comprises 3 mL of the composition in Table 3.

In an alternative embodiment, the cabotegravir for use comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
   wherein, prior to step (a), the human has been administered the first pharmaceutical composition at least once and wherein the first pharmaceutical composition is Apretude.

In an alternative embodiment, the cabotegravir for use comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
   wherein, prior to step (a), the human has been administered the first pharmaceutical composition at least once and wherein the first pharmaceutical composition is Apretude and the second pharmaceutical composition comprises 3 mL of the composition in Table 3.

In an alternative embodiment, the cabotegravir for use comprises the steps of:
(a) administering the first pharmaceutical composition to the human;
(b) 2 months ±7 days after step (a) administering the first pharmaceutical composition to the human;
(c) 2 months ±7 days after step (b) administering the second pharmaceutical composition to the human;
(d) 2 months ±7 days after step (c) administering to the human the second pharmaceutical composition;
(e) administering to the human the second pharmaceutical composition 4 months ±14 days after step (d); and
(f) administering the second pharmaceutical composition every 4 months ±14 days thereafter;
   wherein, prior to step (a), the human has been administered the first pharmaceutical composition at least 3 times, each time 2 months ±7 days apart; and wherein the first pharmaceutical composition is Apretude and the second pharmaceutical composition comprises 3 mL of the composition in Table 3.

The following non-limiting examples illustrate the present invention.

### EXAMPLES

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

### Example 1: Exemplary First Pharmaceutical Composition

Cabotegravir, mannitol, polysorbate 20, PEG 3350, and water for injection were compounded at the levels shown in Table 5 and milled using a wet bead mill. The resulting suspension was filled into 3 mL, USP Type I glass vials at a fill volume of 1.5 mL, the vials are stoppered and sealed, and then terminally sterilized by gamma irradiation.

TABLE 5

| Component | Quantity (mg/mL) |
|---|---|
| Cabotegravir | 200.0 |
| Mannitol | 35.0 |
| Polysorbate 20 | 20.0 |
| Polyethylene Glycol (PEG) 3350 | 20.0 |
| Water for Injection | QS to 1.0 mL |

### Example 2: Exemplary Second Pharmaceutical Composition

TABLE 6a

| Ingredient | Concentration (mg/mL) |
|---|---|
| Cabotegravir, micronized | 400.0 |
| Sodium Carboxymethylcellulose | 5.0 |
| Polysorbate 80 | 4.0 |
| Mannitol | 35.0 |

TABLE 6b

| Ingredient | Concentration (mg/mL) |
|---|---|
| Cabotegravir | 533 (X50 = 4-6 μm) |
| Polysorbate 80 | 5.3 |
| Sodium CMC | 6.7 |
| Mannitol | 46.6 |

Table 6a shows an exemplary second pharmaceutical composition of the invention (pharmaceutical compositions are also described in these examples as “suspensions”), which was made using the following method.

A formulation vehicle was prepared by dissolving/diluting 24.0 g polysorbate 80 (“PS80”) (Croda) in about 300 g water. Separately, 30.0 g sodium carboxymethylcellulose (“NaCMC”) (Ashland, 7L2P) and 210.0 g mannitol (Roquette Freres) were dissolved in 3.4 kg water for injection (WFI). Once the NaCMC and mannitol were dissolved, the PS80 solution was added to the NaCMC-mannitol solution with stirring. The weighing and dilution vessels were rinsed into the compounding vessel with additional water and the compounded vehicle solution was brought to a final weight of 4.50 kg and filtered through a 0.2 μm filter. 1.6 kg Cabotegravir Micronized Free Acid (target X50=5-6 μm particle size) was added to 3.0 kg filtered vehicle and was mixed to form a homogeneous suspension. The compounded suspension was deaerated while stirring until it reached its target batch volume, and the suspension was then filled into vials. The product was lyophilized by freezing at −45° C. for at least 2 hours, annealing at −10° C. for at least 2 hours, refreezing at −45° C. for at least 2 hours (each transition at a ramp rate of +/−1° C./min), primary drying at −5° C. (ramp rate: 1° C./min) for at least 20 hours at approximately 150 mTorr, and secondary drying at 40° C. (ramp rate: 1° C./min) for at least 6 hours at approximately 150 mTorr. The lyophilized vials were backflushed with nitrogen to about 600 Torr, sealed, and sterilized by gamma irradiation at a minimum dose of 25 kGy. The formulation is reconstituted with water for injection (WFI) and briefly shaken to resuspend prior to administration.

The formulation is reconstituted with 1.1 mL WFI and briefly shaken to achieve the formulation in Table 6b.

### Example 3

Example 3 evaluated the safety, tolerability and pharmacokinetics of single-dose administration of the second pharmaceutical composition of the invention in 57 healthy adult participants. The lyophilized formulations of Example 3 are provided in Tables 7 and 8 below:

TABLE 7

| Formulation Components | Cohorts C1 and C2 |
|---|---|
| Component | Quantity (mg/vial) |
| Cabotegravir | 800 |
| Mannitol | 51.8 |
| Polysorbate 80 | 5.9 |
| Sodium Carboxymethylcellulose | 7.4 |
| Water for injection* | q.s. |
| Nitrogen** | q.s. |

*Water is removed during manufacturing process.
**Nitrogen is utilized as a processing aid during vial stoppering.

TABLE 8

| Formulation Components | Cohorts C3-C6 |
|---|---|
| Component | Quantity (mg/vial) |
| Cabotegravir | 800 |
| Mannitol | 70 |
| Polysorbate 80 | 8 |
| Sodium Carboxymethylcellulose | 10 |
| Water for injection* | q.s. |
| Nitrogen** | q.s. |

*Water is removed during manufacturing process.
**Nitrogen is utilized as a processing aid during vial stoppering.

Cabotegravir particle size by diffraction (micron) is described in Table 9 below:

TABLE 9

| Quantile | Micron (μm) |
|---|---|
| X10 | 1.4-3.3 |
| X50 | 3.4-7.5 |
| X90 | 7.6-21.3 |

Vials containing the formulation of Table 7 were reconstituted with 1.7 mL water to achieve suspensions having a cabotegravir concentration of 400 mg/mL. Doses of the formulation of Table 7 were administered in two cohorts: 800 mg (2 ml of 400 mg/mL suspension) via the subcutaneous (SC) abdominal route (Cohort C1; 8 participants) and via the intramuscular (IM) (gluteus medius) route (Cohort C2; 8 participants).

Vials containing the formulation of Table 8 were reconstituted with either 1.7 mL water to achieve suspensions having a cabotegravir concentration of 400 mg/mL or with 1.1 mL water to achieve suspensions having a cabotegravir concentration of 533 mg/ml. Doses of the formulation of Table 8 were administered in three cohorts: 1200 mg (3 mL of 400 mg/ml suspension) via the subcutaneous (SC) abdominal route (Cohort C3; 8 participants), 1200 mg (3 mL of 400 mg/mL suspension) via the intramuscular (IM) (gluteus medius) route (Cohort C4; 8 participants), 1600 mg (3 mL of 533 mg/mL suspension) via the intramuscular (IM) (gluteus medius) route (Cohort C5; 16 participants), 2400 mg (4.5 mL of 533 mg/mL suspension administered as 2.25 mL in each gluteus medius muscle (left and right, respectively) via the IM route (Cohort C6; 9 participants), and 3200 mg (6 mL of 533 mg/mL suspension administered as 3 mL in each gluteus medius muscle (left and right, respectively) via the IM route (Cohort C7; 7 participants). Dose and administration route per cohort is shown in Table 10.

TABLE 10

| Concentration | Cohort | Route | Dose | |
|---|---|---|---|---|
| 400 mg/mL | C1 | SC | 800 mg | (2 mL) |
| | C2 | IM | 800 mg | (2 mL) |
| | C3 | SC | 1200 mg | (3 mL) |
| | C4 | IM | 1200 mg | (3 mL) |
| 533 mg/mL | C5 | IM | 1600 mg | (3 mL) |
| | C6 | IM | 2400 mg | (2x 1200 mg) |
| | C7 | IM | 3200mg | (2x 1600 mg) |

| | | |
|---|---|---|
| C9 | IM | 3200 mg (2x 1600 mg) followed |
| | | by 1600 mg at M 1 |

### Safety and Tolerability

The safety and tolerability profile of the second pharmaceutical compositions, as shown in Tables 11 and 12, and as dosed in Example 3, was acceptable. Adverse events (AEs) occurred in 63-100% of participants. Injection site reactions (ISRs) were the most common AEs and were mostly Grade 1. Overall, fewer ISRs were reported following IM administration compared to SC administration; however, ISRs were predominantly Grade 1. No Grade 4 or serious AEs have been reported in Cohorts C1 to C7.

TABLE 11
Summary of most frequent (≥2 participants across cohorts) AEs (ISR and non-ISR) by cohort

| | Cohort | Cohort | Cohort | Cohort | Cohort | Cohort | Cohort |
|---|---|---|---|---|---|---|---|
| | C1 | C2 | C3 | C4 | C5 | C6 | C7 |
| | 800 mg | 800 mg | 1200 mg | 1200 mg | 1600 mg | 2400 mg | 3200 mg |
| Most Frequent Adverse | SC | IM | SC | IM | IM | IM | IM |
| Events | (N = 8) | (N = 8) | (N = 8) | (N = 8) | (N = 16) | (N = 9) | (N = 7) |
| Any event, n (%) | 8 (100) | 5 (63) | 8 (100) | 8 (100) | 12 (75) | 9 (100) | 7 (100) |
| Any ISR, n (%) | 8 (100) | 3 (38) | 8 (100) | 8 (100) | 11 (69) | 8 (89) | 7 (100) |
| ISRs | | | | | | | |
| Injection site erythema | 8 (100) | 0 | 7 (88) | 0 | 1 (6) | 1 (11) | 0 |
| Injection site pain | 3 (38) | 2 (25) | 5 (63) | 8 (100) | 8 (50) | 7 (78) | 7 (100) |
| Injection site nodule | 6 (75) | 0 | 8 (100) | 0 | 2 (13) | 5 (56) | 1 (14) |
| Injection site swelling | 3 (38) | 2 (25) | 2 (25) | 0 | 3 (19) | 2 (22) | 1 (14) |
| Injection site pruritus | 0 | 0 | 1 (13) | 0 | 0 | 2 (22) | 1 (14) |
| Non-ISRs | | | | | | | |
| Nasopharyngitis/ | 1 (13) | 0 | 0 | 0 | 1 (6) | 0 | 0 |
| pharyngitis | | | | | | | |
| streptococcal | | | | | | | |
| (Viral) upper | 1 (13) | 0 | 2 (25) | 0 | 0 | 0 | 0 |
| respiratory tract | | | | | | | |
| infection | | | | | | | |
| Urinary tract infection | 0 | 0 | 1 (13) | 0 | 0 | 1 (11) | 0 |
| Abdominal | 0 | 2 (25) | 1 (13) | 0 | 1 (6) | 0 | 0 |
| discomfort/abdominal | | | | | | | |
| pain lower/abdominal | | | | | | | |
| pain upper | | | | | | | |
| Diarrhoea | 0 | 1 (13) | 0 | 0 | 1 (6) | 1 (11) | 0 |
| Nausea | 0 | 0 | 0 | 0 | 1 (6) | 0 | 1 (14) |
| Dizziness | 0 | 0 | 1 (13) | 1 (13) | 0 | 0 | 1 (14) |
| Headache | 0 | 1 (13) | 1 (13) | 2 (25) | 0 | 1 (11) | 1 (14) |
| Back pain | 0 | 0 | 0 | 0 | 2 (13) | 0 | 0 |
| Cough | 0 | 1 (13) | 0 | 0 | 1 (6) | 0 | 1 (14) |
| Pollakiuria | 0 | 0 | 0 | 0 | 2 (13) | 0 | 0 |
| Blood creatine | 0 | 0 | 0 | 1 (13) | 0 | 1 (11) | 0 |
| phosphokinase | | | | | | | |
| increased | | | | | | | |

a. ISR duration is not calculated if end date is missing.

TABLE 12
Summary of ISRs

| | Cohort | Cohort | Cohort | Cohort | Cohort | Cohort | Cohort |
|---|---|---|---|---|---|---|---|
| | C1 | C2 | C3 | C4 | C5 | C6 | C7 |
| | SC | IM | SC | IM | IM | IM | IM |
| | 800 mg | 800 mg | 1200 mg | 1200 mg | 1600 mg | 2400 mg | 3200 mg |
| | [2 mL] | [2 mL] | [3 mL] | [3 mL] | [3 mL] | (4.5 mL) | [6 mL] |
| | (n = 8) | (n = 8) | (n = 8) | (n = 8) | (n = 16) | (n = 9 | (n = 7) |
| PARAMETER, N (% OF PARTICIPANTS) | Single injection | | | | | Two injections | |
| Any ISR | 8 (100) | 3 (38) | 8 (100) | 8 (100) | 11 (88) | 8 (89) | 7 (100) |
| Drug-related | 8 (100) | 3 (38) | 8 (100) | 8 (100) | 10 (63) | 8 (89) | 7 (100) |
| Serious | 0 | 0 | 0 | 0 | 0 | 0 | 0 |
| Grade 3 | 0 | 0 | 0 | 0 | 0 | 1 | 0 |
| Grade 4 | 0 | 0 | 0 | 0 | 0 | 0 | 0 |
| Pain | 3 (38) | 2 (25) | 5 (63) | 8 (100) | 8 (50) | 7 (78) | 7 (100) |
| Grade 1 | 3 (38) | 1 (13) | 5 (63) | 8 (100) | 8 (50) | 7 (78) | 5 (71) |
| Grade 2 | 0 | 1 (13) | 0 | 0 | 0 | 0 | 2 (29) |
| Grade 3 | 0 | 0 | 0 | 0 | 0 | 0 | 0 |
| Erythema | 8 (100) | 0 | 7 (88) | 0 | 1 (6) | 1 (11) | 0 |
| Grade 1 | 6 (75) | 0 | 5 (63) | 0 | 1 (6) | 0 | 0 |
| Grade 2 | 2 (25) | 0 | 2 (25) | 0 | 0 | 0 | 0 |
| Grade 3 | 0 | 0 | 0 | 0 | 0 | 1 (11) | 0 |
| Swelling/induration | 3 (38) | 2 (25) | 2 (25) | 0 | 3 (19) | 2 (22) | 1 (14) |
| Grade 1 | 3 (38) | 2 (25) | 2 (25) | 0 | 2 (13) | 1 (11) | 1 (10) |
| Grade 2 | 0 | 0 | 0 | 0 | 1 (6) | 1 (11) | 0 |
| Grade 3 | 0 | 0 | 0 | 0 | 0 | 0 | 0 |
| Nodule | 6 (75) | 0 | 8 (100) | 0 | 2 (13) | 5 (56) | 1 (14) |
| Grade 1 | 6 (75) | 0 | 8 (100) | 0 | 2 (13) | 5 (56) | 1 (14) |
| Grade 2 | 0 | 0 | 0 | 0 | 0 | 0 | 0 |
| Grade 3 | 0 | 0 | 0 | 0 | 0 | 0 | 0 |
| Total ISRs (% of total ISRs) | 21 | 5 | 24 | 9 | 15 | 33 | 21 |
| Grade 1 | 19 (80) | 4 (80) | 22 (92) | 9 (100) | 14 (93) | 29 (88) | 17 (81) |
| Grade 2 | 2 (10) | 1 (20) | 2 (8) | 0 | 1 (7) | 3 (9) | 4 (19) |
| Grade 3 | 0 | | | | | 1 (3) | 0 |
| Grade 4 | 0 | | | | | 0 | 0 |
| Median ISR duration (IQR), | | | | | | | |
| days [n = events with non-missing | | | | | | | |
| end date/total number of events] ^{a} | | | | | | | |
| Pain | n = 3/3 | n = 2/2 | n = 6/6 | n = 8/8 | n = 8/8 | n = 13/13 | n = 14/14 |
| | 7 | 6.5 | 8 | 4.5 | 6 | 7 | 6.5 |
| | (6-12) | (5-8) | (6-15) | (3.5-8.5) | (4-7) | (6-10) | (4-10) |
| Erythema | n = 8/8 | N/A | n = 7/7 | N/A | n = 1/1 | n = 2/2 | N/A |
| | 16 | | 15 | | 4 | 5 | |
| | (10-41) | | (13-31) | | (4-4) | (5-5) | |
| Swelling/induration | n = 3/3 | n = 3/3 | n = 2/2 | N/A | n = 3/3 | n = 4/4 | n = 2/2 |
| | 42 | 5 | 1.5 | | 5 | 38.5 | 2 |
| | (13-63) | (4-43) | (1-2) | | (5-19) | (5-72) | (2-2) |
| Nodule | n = 0/6 | N/A | n = 1/8 | N/A | n = 1/2 | n = 7/9 | n = 1/1 |
| | | | 106 | | 43 | 55 | 10 |
| | | | (106-108) | | (43-43) | (19-106) | (30-10) |

^{a} ISR duration is not calculated if end date is missing.

From this limited short-term safety data on cabotegravir formulation single dose (400 mg/ml and 533 mg/mL), no new systemic or local safety concerns have been observed with IM administration in comparison to the CAB 200 mg/mL suspension approved for use in PrEP (Apretude). To date, single dosing of cabotegravir in the second pharmaceutical composition ranging from 800-3200 mg (800-1600 mg as single injections; 2400 mg as 1200 mg×2 IM injections and 3200 mg as 1600 mg×2 IM injections) shows an acceptable safety profile, including the ISR profile.

Cabotegravir plasma Cmax following a single SC (abdominal) or IM (gluteal) injection of the second pharmaceutical compositions described in Example 3 cohorts 1 to 5 was lower than that following a single IM injection of APRETUDE®, while plasma Cmax following bilateral 2.2+2.3 mL IM gluteal injections (4.5 mL total) was comparable to that following a single IM injection of APRETUDE®. Based on observed PK data for cohorts 1 to 5 in Example 9, t½ following these cohorts of SC and IM injection is predicted to be >6×(SC injections) and >2×(IM injections) the t_{1/2} of APRETUDE® (Q2M cabotegravir 200 mg/mL), respectively. There are not enough data to determine the t_{1/2} following administration of the cabotegravir pharmaceutical composition described in cohorts 6 or 7.

TABLE 13
Plasma Pharmacokinetic and Safety Results in Healthy Adult Participants Cohorts C1-C7

| | Cohort | Cohort | Cohort | Cohort | Cohort | Cohort | Cohort |
|---|---|---|---|---|---|---|---|
| | C1 | C2 | C3 | C4 | C5 | C6 | C7 |
| | (2 mL) | (2 mL) | (3 mL) | (3 mL) | (3 mL) | (4.5 mL) | (6 mL) |
| | SC | IM | SC | IM | IM | IM | IM |
| Parameter | (N = 8) | (N = 8) | (N = 8) | (N = 8) | (N = 16) | (N = 9) | (N = 7) |
| Cmax, | 0.7 (35.1) | 1.8 (43.0) | 0.9 (32.6) | 1.8 (130.7) | 1.9 (85.3) | 4.1 (40.4) | * |
| μg/mL^{a} | | | | | | | |
| Grade 1 | 6 (75) | 2 (25) | 6 (75) | 8 (100) | 10 (63) | 7 (78) | 5 (71) |
| ISRs, n (%) | | | | | | | |
| Grade 2 | 2 (25) | 1 (13) | 2 (25) | 0 | 1 (6) | 0 | 2 (29) |
| ISRs, n (%) | | | | | | | |
| Grade ≥3 | 0 | 0 | 0 | 0 | 0 | 1 (11) | 0 |
| ISRs, n (%) | | | | | | | |

^{a}Value reported as geometric mean (% CVb).
*not yet calculable.

### Example 4

A proposed clinical study consists of loading injections of a first pharmaceutical composition of cabotegravir (CAB) (200 mg/mL) 600 mg intramuscularly (IM) (described herein as CAB LA), having the composition Table 5, which is the formulation of Apretude; followed by a second pharmaceutical composition of cabotegravir 533 mg/ml, 3 mL, 1600 mg IM injections having the composition in Table 6b (described herein as CAB ULA) to participants naïve to cabotegravir. Specifically, CAB LA 600 mg will be administered on Day 1 and Month 1 of the study. Subsequently, CAB ULA 1600 mg will be administered at Month 3, Month 5 and every 4 months thereafter. All injections are 3 mL.

The target exposure outcome for CAB ULA is that at least 90% of individuals achieve plasma CAB Ctau≥10th percentile of steady state (SS) plasma trough concentration (Ctau) observed in the HPTN 083 and HPTN 084 registrational clinical studies. In both registrational studies, CAB LA (200 mg/mL) was well-tolerated and superior to daily oral tenofovir disoproxil fumarate (TDF)/emtricitabine (FTC) in preventing HIV acquisition. In both registrational studies, CAB LA was administered intramuscularly by a healthcare provider once every 2 months (Q2M) with the first 2 injections given 1 month apart. There were few confirmed incident HIV infections in the CAB arms of HPTN 083 and HPTN 084. Exposures in participants with confirmed incident HIV infections while receiving CAB LA injections (n=4 in HPTN 083, n=1 in HPTN 084) were overlapping with those who did not acquire HIV infections during the blinded phase of the studies. Multiple other factors, including concomitant sexually transmitted infection (STI), mucosal trauma, viral phenotype, and inoculum size, may be associated with the occurrence of HIV infections (Error! Reference source not found., 2020).

The 10th percentile of CAB SS Ctau was 1.05 μg/mL in HPTN 083 (n=170) and 1.39 μg/mL in HPTN 084 (n=358), which was calculated based on all SS Ctau values from all participants with post-injection PK data who received CAB injections Q8W before discontinuation (if any) and did not acquire HIV. Ctau was defined as concentrations collected at nominal Q8W injection visits. The start of steady state was determined to be Week 9 in HPTN 083 and Week 17 in HPTN 084. A total of 794 SS Ctau values from 170 participants in HPTN 083 and 1427 SS Ctau values from 358 participants in HPTN 084 were included in the calculations. The 10th percentiles of the first Ctau following the first injection was 0.672 μg/mL in HPTN 083 (n=114) and 0.683 μg/mL in HPTN 084 (n=336).

The objective of this study is to confirm that the plasma trough cabotegravir concentration following administration of CAB ULA IM Q4M gluteal injections, following the loading dose with CAB LA Q2M, are equal to or higher than those observed in Q8W registrational studies (HPTN 083 and HPTN 084), separately by sex at birth subgroups. The study will assess evaluable steady state CAB trough concentrations at or above the target of 1.05 μg/mL for people assigned male at birth (AMAB) and 1.39 μg/mL for people assigned female at birth (AFAB) once steady-state has been achieved. It is predicted that approximate CAB steady state will be achieved by Month 13, and the Month 13 Ctau will achieve the target SS Ctau observed in HPTN 083 and HPTN 084.

Based on the robust efficacy seen with CAB LA PrEP in both HPTN 083 and HPTN 084, a CAB ULA regimen that achieves similar trough concentrations but with less frequent dosing would provide similar efficacy in preventing HIV infection. In addition, a CAB ULA formulation achieving the similar peak concentrations or maximal concentrations (Cmax) as that observed in HPTN 083 and HPTN 084 is anticipated to convey the same systemic safety.

Each injection of CAB LA or CAB ULA will be a single 3 mL injection administered intramuscularly in the gluteus medius. To extend the interval between injections at the same site, injections should be rotated between the right and left gluteus medius at alternate injections. The time and location of injection will be captured in the eCRF. The sequence of injections are as follows:
Day 1 (Baseline): Initial injection of CAB LA (600 mg)·
Month 1: Second injection of CAB LA (600 mg)·
Month 3: First injection of CAB ULA (1600 mg)·
Months 5, 9, 13, 17, 21, 25, and 29: Q4M maintenance dosing of CAB ULA (1600 mg).

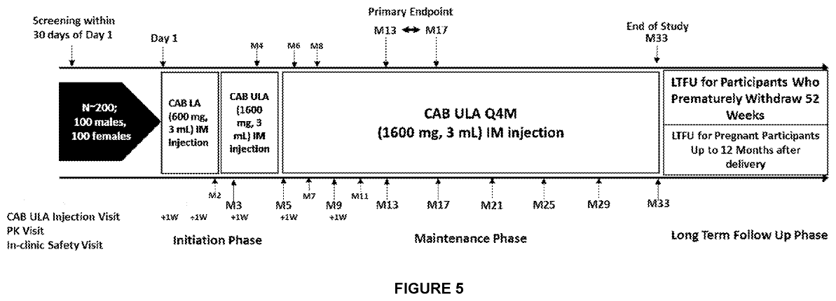

The primary endpoint will be the proportion of evaluable CAB trough concentrations above the 10th percentile target (1.05 μg/mL for people assigned male at birth and for men and 1.39 μg/mL for people assigned female at birth) once steady state has been achieved, but no earlier than Month 13. FIG. 5 shows a schematic of the study design.

CAB ULA 1600 mg has been administered in 16 individuals in Example 3 Cohort C5. Preliminary parameters are summarized in Table 14. Median (range) CAB concentrations 16-weeks post injection, representing a 4-monthly dosing interval, was 0.952 μg/mL (0.341-2.14). Apparent terminal phase t½ could not be measured directly due to the limited amount of data available in the terminal phase and therefore is likely to be longer than 24 weeks. Assuming ~2.5-fold accumulation based on approximate terminal phase absorption rate limited half-life of ~22 weeks, steady state CAB trough concentrations following 1600 mg Q4M in these participants are estimated to be 2.38 μg/mL (0.853-5.35), at or above geometric mean (5^{th}, 95^{th} percentile) CAB trough concentrations following CAB LA Q2M of 2.01 μg/mL (0.64, 4.73).

TABLE 14
Preliminary Summary of plasma CAB parameters
following administration of Cabotegravir
1600 mg (CAB ULA) (Cohort 5, n = 16)

| Data through | Cmax | Tmax | C, 16 wk |
|---|---|---|---|
| (week) | (μg/mL) | (day) | (μg/mL) |
| 24-36 | 1.62 (0.554, | 28.0 (4.00, | 0.952 (0.341, |
| | 7.93) | 167) | 2.14) |

### PopPK Models

A cabotegravir population pharmacokinetic (PopPK) model for CAB LA (APRETUDE®) was built and updated based on PK data following CAB 200 mg/mL IM injections collected in 19 historical studies. The CAB LA model was used as a starting point for CAB ULA (Cab 533 mg/mL Q4M) model development. The systemic cabotegravir parameters, which are independent of formulation (clearance and volume), and their inter-individual variabilities (IIV), were fixed to the CAB LA PopPK model parameter estimates. Parameters with the potential to be influenced by formulation, such as absorption parameters and their IIVs, were re-estimated based on CAB ULA plasma concentration data from the study shown in Example 3 following the administration of single doses of CAB ULA, from 800 to 3200 mg, in a total of 48 healthy participants.

The CAB LA PopPK model was used to simulate concentration-time profiles for 10,000 virtual subjects in different Apretude loading scenarios. Covariates including body weight and BMI were resampled 5000 times from the distribution of these covariates for males and females, respectively, generating 5000 virtual male subjects and 5000 virtual female subjects. Individual PK parameters of the virtual subjects were calculated using subject-specific covariates and using the population parameter estimates, subject-specific NONMEM inter-individual errors (ETAs) sampled from the distributions that are decided by the estimated variance-covariate matrix of between-subject variability from the final population PK model. Concentration-versus-time profiles of the virtual subjects were calculated using the individual PK parameters. Residual variability (EPS) was included in the simulation. Median and 10th and 90th percentiles (80% prediction interval) of the simulated concentration-versus-time profiles of the virtual males and females were calculated.

The concentration at the end of the CAB LA dosing interval (ie concentration right before the first simulated administration of CAB ULA) was used as the starting concentration (concentration at time zero) for incorporation of the CAB ULA model. At that time, the CAB ULA PopPK model was used to simulate concentration-time profiles for the previously-defined 10,000 virtual subjects (using the absorption parameters determined by the CAB ULA PopPK model) in different CAB ULA loading scenarios.

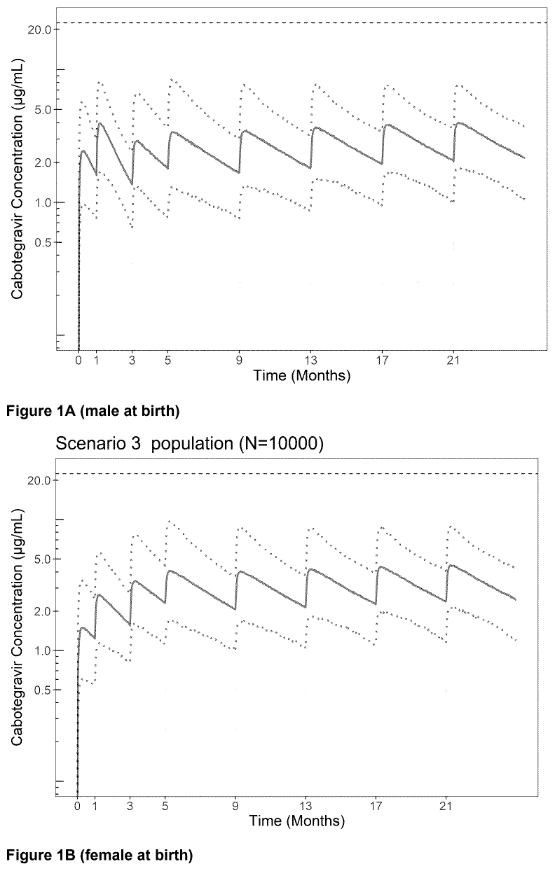

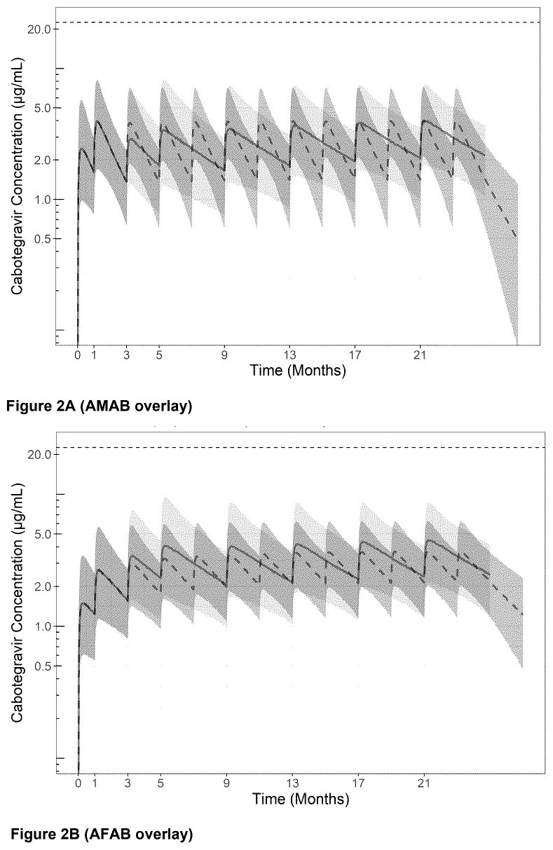

An APRETUDE® load was employed to enable the use of CAB ULA 1600 mg in a modified loading strategy to abrogate the need for a large CAB ULA load. To that end, scenarios were developed with varying contributions of CAB LA 600 mg and CAB ULA 1600 mg. Two doses of CAB LA (Day 1 and Month 1), followed by CAB ULA 1600 mg at Month 3 and then Month 5, is predicted to achieve CAB plasma trough concentrations following CAB ULA administration similar to that following APRETUDE® administration. Simulations from virtual administration of this regimen are shown in FIG. 1A (AMAB) and 1B (AFAB) Results of this simulated regimen are shown in FIG. 2, overlaid with the results of administration of the APRETUDE® regimen for the duration of a study. Predicted plasma CAB exposures are similar between those arising from the approved APRETUDE® regimen and those arising from the regimen that incorporates CAB ULA.

Specifically, individuals are not expected to exceed either the plasma CAB concentration long-term threshold of 13.1 μg/mL, which is the median Cmax following oral 60 mg once daily for 2 years in the LAI116482 (LATTE) trial, or the short-term threshold of 22.5 μg/mL established following supratherapeutic dosing of oral CAB in the dedicated TQT study (LAI117009), which was not associated with any toxicity. Overall, predicted targets are expected to be achieved in ~90% of participants following CAB LA initiation at day 1 and month 1 followed by CAB ULA 1600 mg at month 3 and Q4M from month 5 onwards.

The simulations show that 2 doses of Apretude are needed to provide sufficient sustainable plasma exposure to CAB before less frequent ULA (533 mg/mL, 3 mL dose) dosing (2-month interval between first and second injection of Cab ULA, followed by Q4M), This data supports that patients who are cabotegravir naïve or patients who are already taking APRETUDE® can start a dosing regimen of CAB ULA.

### Example 5

An additional study has been designed to assess the pharmacokinetics, safety, and tolerability of switching to a second pharmaceutical formulation (described in Example 2 and described herein as CAB ULA) 533 mg/mL cabotegravir Q4M in the same healthy adult participants who previously received a first pharmaceutical composition of cabotegravir (CAB) (200 mg/mL) described herein as CAB LA), which is the formulation disclosed in Example 1 i.e. the formulation of Apretude) administered as 600 mg intramuscular (IM) injections for 7 months according to the approved 2-monthly schedule. The primary endpoint will evaluate if plasma trough concentrations of Q4M CAB ULA IM gluteal injections after switching from Q2M CAB LA IM gluteal injections are equal to or higher than those observed in the CAB LA/pre-switch phase. Safety, PK, and efficacy are well established for CAB LA and thus, a CAB ULA regimen that achieves similar concentrations is anticipated to achieve the same safety and antiviral efficacy.

The CAB LA dosing schedule is consistent with the approved dosing regimen for CAB 200 mg for PrEP that was assessed in HTPN 084 and 083 studies which has demonstrated significant antiviral efficacy and protection in clinical studies with an acceptable safety profile. The approved CAB LA (200 mg/mL) 600 mg IM PrEP regimen will be administered Q2M for 7 months. Specifically, participants will receive a 600 mg initiation injection on Day 1, followed by four 600 mg maintenance injections Q2M from Month 1 to Month 7. The weighted average CAB LA half-life estimated for this study based on population PK modelling is ~7.4 weeks (70:30 population of males (t½ 5.6 weeks) and females (t½ 11.5 weeks), and steady state should be achieved by the overall population in the study at the end of the CAB LA Phase at Month 9.

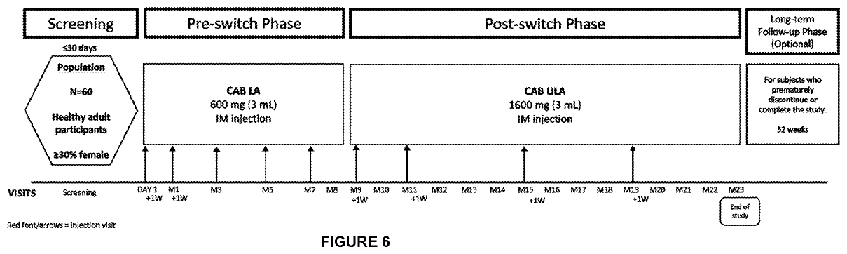

Starting two months following the final CAB LA injection, CAB ULA 1600 mg (533 mg/mL) (as defined in Example 2) injections will be administered at 4 visits, specifically at Month 9, Month 11, Month 15 and Month 19. Observed data from Example 3 and modelling and simulation suggest that steady state plasma CAB concentrations will be reached at the end of the ULA injection phase (Month 23). FIG. 6 shows a schematic of the study design.

Median Cmax throughout the entire Q4M regimen is predicted to remain below the long-term safety threshold of 13.1 μg/mL, which is the observed median steady state Cmax in Study LAI116482 (LATTE) following daily oral CAB 60 mg for 2 years. No significant safety concerns were observed in the LATTE study across all oral doses of 10 mg, 30 mg, or 60 mg once daily. Further, no PK/pharmacodynamic (PD) relationship for safety has been established for CAB 200 mg/mL, and as the projected CAB ULA median Cmax in this study is below 13.1 μg/mL, it is anticipated that a different systemic safety profile is unlikely. In addition, the probability of exceeding the short-term safety threshold of 22.5 μg/mL, the geometric mean Cmax following CAB 150 mg q12 h×3 doses in the dedicated TQT study where no safety signal was identified, is negligible.

CAB ULA (533 mg/mL) 1600 mg has been administered in 16 individuals in Study 218012 Cohort 5. Preliminary parameters are summarized in 14. Median (range) CAB concentrations 16-weeks post injection, representing a 4-monthly dosing interval, was 0.952 μg/mL (0.341-2.14). Apparent terminal phase t½ could not be measured directly due to the limited amount of data available in the terminal phase and therefore is likely to be longer than 24 weeks. Assuming ~2.5-fold accumulation based on approximate terminal phase absorption rate limited half-life of ~22 weeks, steady state CAB trough concentrations following 1600 mg Q4M in these participants are estimated to be 2.38 μg/mL (0.853-5.35), at or above geometric mean (5th, 95th pct) CAB trough concentrations following CAB LA Q2M of 2.01 μg/mL (0.64, 4.73).

A CAB ULA population PK model has been developed using data following IM injections across multiple dosing cohorts in the study in Example 3 (PK model discussed above in Example 4). Simulations were generated to predict exposures following administration of CAB ULA after switching from CAB LA. The predicted CAB concentration-time profile for the proposed regimen is shown in Error! Reference source not found . . . . The lower dashed line represents the observed 5th percentile following initiation injection (0.45 μg/mL) and the upper dashed line shows the safety threshold (22.5 μg/mL). Solid lines represent simulated median (middle line) and 10th and 90th percentile (upper and lower line) CAB concentrations.CAB ULA Q4M maintains or exceeds CAB LA Q2M concentrations after switching (Error! Reference source not found., Table 15). CAB trough concentrations are predicted to be 28% higher at Month 23 after the third full Q4M ULA injection when compared to Month 9 following the fourth full Q2M CAB LA injection.

TABLE 15
Predicted CAB troughs following CAB LA and CAB ULA
CAB trough (μg/mL)

| | | CAB ULA:CAB LA |
|---|---|---|
| CAB LA M 9 | CAB ULA M 23 | GSLM ratio (CI) |
| 1.68 | 2.26 | 1.28 |
| [0.77, 2.97] | [1.03, 3.72] | (1.27, 1.30) |
| {0.57, 3.45} | {0.79, 4.3} | |
| 1.56 (63.6%) | 2.01 (58.2%) | |

Notes:
M 9 and M 23 CAB troughs summarized as Median [10^{th}, 90^{th}] and {5^{th}, 95^{th}} percentiles; geomean (CVb %)

All of the U.S. patents, U.S. patent application publications, U.S. patent applications, foreign patents, foreign patent applications, and non-patent publications referred to in this specification are incorporated herein by reference, in their entirety to the extent not inconsistent with the present description.

From the foregoing it will be appreciated that, although specific embodiments have been described herein for purposes of illustration, various modifications may be made without deviating from the spirit and scope of the disclosure. Accordingly, the disclosure is not limited except as by the appended claims.

### Embodiments

In the below list, embodiments are denoted as ‘E’.

E1. A method of preventing HIV infection in a human, comprising:
(a) administering a first pharmaceutical composition to the human;
(b) subsequently administering the first pharmaceutical composition to the human; and
(c) subsequently administering a second pharmaceutical composition to the human;
wherein the first pharmaceutical composition comprises about 200 mg/ml of cabotegravir, and
wherein the second pharmaceutical composition comprises about 533 mg/ml of cabotegravir.

E2. The method according to E1, wherein step (c) occurs 2 months ±7 days after step (b).

E3. The method according to E 2, wherein step (c) occurs 2 months after step (b).

E4. The method according to E 2 or 3, further comprising:
(g) administering to the human the second pharmaceutical composition 2 months ±7 days after step (c).

E5. The method according to E 4, wherein in step (d) the administering occurs 2 months after step (c).

E6. The method according to E 4 or 5, further comprising:
(e) administering to the human the second pharmaceutical composition 4 months ±14 days after step (d).

E7. The method according to E 6, wherein in step (e) the administering occurs 4 months after step (d).

E8. The method according to E 6 or E 7, further comprising:
(f) administering the second pharmaceutical composition every 4 months ±14 days thereafter.

E9. The method according to E 8, wherein the administering in step (f) occurs every 4 months thereafter.

E10. The method according to any preceding embodiment wherein step (b) occurs 1 month ±7 days after step (a).

E11. The method according to E 10, wherein step (b) occurs 1 month after step (a).

E12. The method according to E 10 or 11, wherein prior to step (a) the human has never previously been administered the first pharmaceutical composition.

E13. The method according to any of E 1 to 9, wherein step (b) occurs 2 months ±7 days after step (a).

E14. The method according to E 13, wherein step (b) occurs 2 months after step (a).

E15. The method according to E 13 or 14, wherein prior to step (a) the human has been administered the first pharmaceutical composition at least 3 times.

E16. The method according to E 15, wherein prior to step (a), the human has been administered the first pharmaceutical composition at least 3 times, each time 2 months ±7 days apart.

E17. The method according to E 16, wherein prior to step (a), the human has been administered the first pharmaceutical composition at least 3 times, each time 2 months apart.

E18. The method according to any preceding embodiment, wherein the first pharmaceutical composition comprises about 600 mg of cabotegravir.

E19. The method according to any preceding embodiment, wherein the first pharmaceutical composition is APRETUDE®.

E20. The method according to any preceding embodiment, wherein the second pharmaceutical composition comprises about 1600 mg of cabotegravir.

E21. The method according to any preceding embdiment, wherein the second pharmaceutical composition further comprises polysorbate 80 and sodium carboxymethylcellulose (CMC).

E22. The method according to E 21, wherein the pharmaceutical composition further comprises mannitol.

E23. The method according to E 21 or 22, wherein the ratio of cabotegravir:polysorbate 80:sodium CMC is 400:4:5.

E24. The method according to any preceding embodiment, wherein the particle size of the second pharmaceutical composition is between (and including) 4 and 6 μm.

E25. Cabotegravir for use in the prevention of HIV infection in a human, the use comprising:
(h) administering a first pharmaceutical composition to the human;
(i) subsequently administering the first pharmaceutical composition to the human; and
(j) subsequently administering a second pharmaceutical composition to the human;
wherein the first pharmaceutical composition comprises about 200 mg/ml of cabotegravir, and
wherein the second pharmaceutical composition comprises about 533 mg/ml of cabotegravir.

E26. Cabotegravir for use according to E 25, wherein step (c) occurs 2 months ±7 days after step (b).

E27. Cabotegravir for use according to E 26, wherein step (c) occurs 2 months after step (b).

E28. Cabotegravir for use according to E 26 or 27, further comprising:
(n) administering to the human the second pharmaceutical composition 2 months ±7 days after step (c).

E29. Cabotegravir for use according to E 28, wherein in step (d) the administering occurs 2 months after step (c).

E30. Cabotegravir for use according to E 28 or 29, further comprising:
(e) administering to the human the second pharmaceutical composition 4 months ±14 days after step (d).

E31. Cabotegravir for use according to E 30, wherein in step (e) the administering occurs 4 months after step (d).

E32. Cabotegravir for use according to E 30 or E 31, further comprising:
(f) administering the second pharmaceutical composition every 4 months ±14 days thereafter.

E33. Cabotegravir for use according to E 32, wherein the administering in step (f) occurs every 4 months thereafter.

E34. Cabotegravir for use according to E 33, wherein step (b) occurs 1 month ±7 days after step (a).

E35. Cabotegravir for use according to E 34, wherein step (b) occurs 1 month after step (a).

E36. Cabotegravir for use according to any of E33 to 35, wherein prior to step (a) the human has never previously been administered the first pharmaceutical composition.

E37. Cabotegravir for use according to any of E 25 to 36, wherein step (b) occurs 2 months ±7 days after step (a).

E38. Cabotegravir for use according to E 37, wherein step (b) occurs 2 months after step (a).

E39. Cabotegravir for use according to E 37 or 38, wherein prior to step (a) the human has been administered the first pharmaceutical composition at least 3 times.

E40. Cabotegravir for use according to E 39, wherein prior to step (a), the human has been administered the first pharmaceutical composition at least 3 times, each time 2 months ±7 days apart.

E41. Cabotegravir for use according to E 40, wherein prior to step (a), the human has been administered the first pharmaceutical composition at least 3 times, each time 2 months apart.

E42. Cabotegravir for use according to any of 25 to 41, wherein the first pharmaceutical composition comprises about 600 mg of cabotegravir.

E43. Cabotegravir for use according to any of E 25 to 42, wherein the first pharmaceutical composition is APRETUDE®.

E44. Cabotegravir for use according to any of E 25 to 43, wherein the second pharmaceutical composition comprises about 1600 mg of cabotegravir.

E45. Cabotegravir for use according to any of E 25 to 44, wherein the second pharmaceutical composition comprises polysorbate 80 and sodium carboxymethylcellulose (CMC).

E46. Cabotegravir for use according to E 45, wherein the pharmaceutical composition further comprises mannitol.

E47. Cabotegravir for use according to E45 or 46, wherein the ratio of cabotegravir:polysorbate 80:sodium CMC is 400:4:5.

E48. Cabotegravir for use according to any of E 25 to 47, wherein the particle size of the second pharmaceutical composition is between (and including) 4 and 6 μm.

1. A method of preventing HIV infection in a human, comprising:
(a) administering a first pharmaceutical composition to the human;
(b) subsequently administering the first pharmaceutical composition to the human; and
(c) subsequently administering a second pharmaceutical composition to the human;
wherein the first pharmaceutical composition comprises about 200 mg/ml of cabotegravir, and
wherein the second pharmaceutical composition comprises about 533 mg/mL of cabotegravir and wherein step (c) occurs 2 months ±7 days after step (b).

2. The method according to claim 1, further comprising:
(d) administering to the human the second pharmaceutical composition 2 months ±7 days after step (c).

3. The method according to claim 2, further comprising:
(e) administering to the human the second pharmaceutical composition 4 months ±14 days after step (d).

4. The method according to claim 3, further comprising:
(f) administering the second pharmaceutical composition every 4 months ±14 days thereafter.

5. The method according to claim 1 wherein step (b) occurs 1 month ±7 days after step (a) and prior to step (a) the human has never previously been administered the first pharmaceutical composition.

6. The method according to claim 1, wherein step (b) occurs 2 months ±7 days after step (a) and wherein prior to step (a), the human has been administered the first pharmaceutical composition at least 3 times, each time 2 months ±7 days apart.

7. The method according to claim 1, wherein the first pharmaceutical composition is APRETUDE®.

8. The method according to claim 1, wherein the second pharmaceutical composition comprises about 1600 mg of cabotegravir.

9. The method according to claim 1, wherein the second pharmaceutical composition further comprises polysorbate 80 and sodium carboxymethylcellulose (CMC) and wherein the ratio of cabotegravir:polysorbate 80:sodium CMC is 400:4:5.

10. The method according to claim 1, wherein the particle size of the second pharmaceutical composition is between (and including) 4 and 6 μm.

11.-20. (canceled)

21. The method according to claim 2 wherein step (b) occurs 1 month ±7 days after step (a) and prior to step (a) the human has never previously been administered the first pharmaceutical composition.

22. The method according to claim 2, wherein step (b) occurs 2 months ±7 days after step (a) and wherein prior to step (a), the human has been administered the first pharmaceutical composition at least 3 times, each time 2 months ±7 days apart.

23. The method according to claim 2, wherein the first pharmaceutical composition is APRETUDE®.

24. The method according to claim 2, wherein the second pharmaceutical composition comprises about 1600 mg of cabotegravir.

25. The method according to claim 2, wherein the second pharmaceutical composition further comprises 80 and sodium polysorbate carboxymethylcellulose (CMC) and wherein the ratio of cabotegravir:polysorbate 80:sodium CMC is 400:4:5.

26. The method according to claim 2, wherein the particle size of the second pharmaceutical composition is between (and including) 4 and 6 μm.

27. The method according to claim 3 wherein step (b) occurs 1 month ±7 days after step (a) and prior to step (a) the human has never previously been administered the first pharmaceutical composition.

28. The method according to claim 3, wherein step (b) occurs 2 months ±7 days after step (a) and wherein prior to step (a), the human has been administered the first pharmaceutical composition at least 3 times, each time 2 months ±7 days apart.

29. The method according to claim 3, wherein the first pharmaceutical composition is APRETUDE®.

30. The method according to claim 3, wherein the second pharmaceutical composition comprises about 1600 mg of cabotegravir.

31. The method according to claim 3, wherein the second pharmaceutical composition further comprises 80 and polysorbate sodium carboxymethylcellulose (CMC) and wherein the ratio of cabotegravir:polysorbate 80:sodium CMC is 400:4:5.

32. The method according to claim 3, wherein the particle size of the second pharmaceutical composition is between (and including) 4 and 6 μm.
